(19) United States
(12) Patent Application Publication
(10) Pub. No.: US 2020/0000766 A1
(43) Pub. Date: Jan. 2, 2020

(54) COMPOSITIONS AND METHODS FOR AGONIZING THE CB2 RECEPTOR

(71) Applicant: CANOPY GROWTH CORPORATION, Smith Falls (CA)

(72) Inventors: Brian Geoffrey REID, Louisville, CO (US); Jonathan Michael COOPER, Denver, CO (US)

(21) Appl. No.: 16/456,078

(22) Filed: Jun. 28, 2019

Related U.S. Application Data
(60) Provisional application No. 62/691,343, filed on Jun. 28, 2018.

(51) Int. Cl. A61K 31/352; A61K 45/06; A61K 31/045; A61K 31/01; A61K 31/215

(52) U.S. Cl. CPC A61K 31/352; A61K 45/06; A61K 31/215; A61K 31/01; A61K 31/045

(57) ABSTRACT

Disclosed herein are new compositions comprising a purified cannabinoid and a purified terpene. In one embodiment, the compositions comprise one or more purified cannabinoids. In one embodiment, the compositions comprise one or more purified cannabinoids in combination with one or more purified terpenes. In one embodiment, the compositions comprise unnaturally occurring ratios. In one embodiment, the compositions comprise unnaturally occurring concentrations. In one embodiment, the compositions comprise unexpected and/or synergistic effects.

### CROSS-REFERENCE TO RELATED APPLICATION

This application claims priority to U.S. Provisional Patent Application Ser. No. 62/691,343, filed on Jun. 28, 2018, which is hereby incorporated by reference.

### TECHNICAL FIELD

This disclosure relates to the cannabis industry. In particular, this disclosure relates to methods of treatment comprising administering compositions and formulations comprising new, purposefully engineered combinations of purified CBC with or without one or more additional purified compounds.

### BACKGROUND

The word “cannabis” refers to a genus of flowering plants. Plants of genus cannabis include several species, including Cannabis sativa, Cannabis indica, and Cannabis ruderalis. There is a long history of cultivating plants of genus cannabis for hemp fibers, seeds and seed oils, medicinal purposes, and recreational activities.

According to some accounts, cannabis is composed of at least 483 known chemical compounds, which include cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, terpenes, non-cannabinoid phenols, vitamins, and pigments.

Cannabinoids are of particular interest for research and commercialization. Most extractions of cannabis plant matter aim to extract cannabinoids, particularly in their neutral and/or acid forms. For example, tetrahydrocannabinol (THC) and/or tetrahydrocannabinolic acid (THCA) may be extracted using known techniques, and THCA may be converted to THC by decarboxylation. THC is considered useful for relieving pain, treating glaucoma, and relieving nausea. THC is also gaining popularity as a recreational drug substance. Usually, cannabinoids are extracted from the cannabis plant as part of a crude mixture or whole plant extract, which also includes other chemical compounds found in the cannabis plant.

Amounts of the chemical compounds can vary widely among samples and batches of extracts. There exists a considerable need for compositions containing isolated, purified forms of these compounds in particular combinations not ordinarily found in nature. Some of the less common cannabinoids have neither been isolated nor studied alone or in any combination in physiologically relevant assays to assess their therapeutic potential. Additionally, there exists a considerable need for compositions having purposefully engineered, repeatable, consistent, and dependable ratios of purified cannabinoids, as well as for compositions that specifically combine a cannabinoid with a purified terpene.

There also is a considerable need for methods of activating or agonizing particular cannabinoid receptors (e.g., CB2) considered important to treating diseases and conditions that would benefit from selectively agonizing that receptor, including, for example, inflammation, inflammatory diseases, and neuropathic pain.

Additionally, there is a considerable need for compositions and methods comprising an isolated, purified cannabinoid combined with an analgesic, wherein the cannabinoid and analgesic are synergistic in their pain-relieving effects, such as for example, compositions comprising isolated, purified, CBC and an analgesic.

There exists a need for new methods comprising administering purified cannabis components. In particular, there exists a need for methods of agonizing or activating particular cannabinoid receptors, such as the CB2 receptor comprising administering isolated, purified CBC, alone or in combination with other cannabinoid(s) and/or terpene(s), and/or an analgesic.

### BRIEF DESCRIPTION OF THE DRAWINGS

FIG. 1 is a data plot of the results from a first independent experiment showing the significantly greater potency of CBC for CB2 receptors, as described in Example 1.

FIG. 2 is a data plot of the results from a second independent experiment showing the significantly greater potency of CBC for CB2 receptors, as described in Example 1.

### DETAILED DESCRIPTION

Disclosed herein are new methods of agonizing the CB2 receptor comprising administering one or more purified cannabinoids and/or one or more purified terpenes. In one embodiment the disclosed methods comprise administering purified CBC. In one embodiment the disclosed methods comprise administering a first purified cannabinoid and a first purified terpene. In one embodiment of this disclosure, the methods comprise administering a first purified cannabinoid and a second purified cannabinoid. In one embodiment, the methods comprise administering a first purified cannabinoid, a second purified cannabinoid, and a first purified terpene. In one embodiment of this disclosure, the methods comprise administering a first purified cannabinoid, a first purified terpene, and a second purified terpene. In one embodiment, the methods disclosed herein comprise administering a composition comprising purified CBC to a subject identified as needing agonism of the CB2 receptor. In one embodiment, the methods disclosed herein comprise administering a composition comprising purified CBC to a subject in need of treatment for pain. In one embodiment, CBC is administered together with an analgesic.

Disclosed herein are methods of agonizing the CB2 receptor, comprising administering a composition comprising purified CBC. In one embodiment, the method further comprises administering an analgesic. In one embodiment, the method further comprises administering a second cannabinoid. In one embodiment, the disclosed methods comprise concurrently administering a cannabinoid and an analgesic. In one embodiment, the disclosed methods comprise administering a dosage formulation comprising a cannabinoid and an analgesic. In one embodiment the disclosed methods comprise administering purified CBC. In one embodiment, the disclosed methods comprise administering concurrently CBC and an analgesic. In one embodiment, the disclosed methods comprise administering concurrently CBC and a second cannabinoid. In one embodiment, the disclosed methods comprise administering concurrently CBC and a terpene.

The methods disclosed herein comprise administering compositions that are purposefully formulated to provide man-made, non-naturally occurring combinations, concentrations, and/or ratios of compounds derived from cannabis plants.

In one embodiment, the compositions disclosed herein include a non-naturally occurring ratio of a first purified cannabinoid and a first purified terpene. In one embodiment, the compositions disclosed herein include a non-naturally occurring concentration of a first purified cannabinoid and a first purified terpene.

In one embodiment, the compositions disclosed herein include a non-naturally occurring ratio of a first purified cannabinoid and a second purified cannabinoid. In one embodiment, the compositions disclosed herein include a non-naturally occurring concentration of a first purified cannabinoid and a second purified cannabinoid.

In one embodiment, the compositions disclosed herein include a non-naturally occurring ratio of a first purified cannabinoid, a second purified cannabinoid, and a first purified terpene. In one embodiment, the compositions disclosed herein include a non-naturally occurring concentration of a first purified cannabinoid, a second purified cannabinoid, and a first purified terpene.

In one embodiment, the compositions disclosed herein include a non-naturally occurring ratio of a first purified cannabinoid, a second purified cannabinoid, and a third purified cannabinoid. In one embodiment, the compositions disclosed herein include a non-naturally occurring concentration of a first purified cannabinoid, a second purified cannabinoid, and a third purified cannabinoid.

In one embodiment, the compositions used in the methods disclosed herein include a non-naturally occurring ratio of a first purified terpene, a second purified terpene, and a first purified cannabinoid. In one embodiment, the compositions used in the methods disclosed herein include a non-naturally occurring concentration of a first purified terpene, a second purified terpene, and a first purified cannabinoid.

In one embodiment of this disclosure, the compositions comprise an unnaturally occurring ratio of 10:1-1:1 of a purified cannabinoid to purified terpene.

In one embodiment of this disclosure, the compositions comprise an unnaturally occurring ratio of 10:1-5:1 of a purified cannabinoid to purified terpene.

In one embodiment of this disclosure, the compositions comprise an unnaturally occurring ratio of greater than 10:1 of a purified cannabinoid to purified terpene.

Disclosed herein are compositions comprising:
a first purified cannabinoid; and
a compound chosen from a second purified cannabinoid or a first purified terpene or an analgesic, wherein the said composition is substantially free from cellulose.

As used within the context of this disclosure, the term “purified” means isolated and/or separated from other compounds, formulations, compositions, matter, and/or mass. In one embodiment, the term “purified” refers to a cannabinoid that is separated from the plant matter from which it was derived.

In one embodiment, the term “purified” refers to a cannabinoid (a “purified cannabinoid”) that is separated from other cannabinoids that were present in the plant matter from which it was derived. In one embodiment, the term “purified” refers to a cannabinoid (a “purified cannabinoid”) that is separated from terpenes that were present in the plant matter from which it was derived. In one embodiment, the term “purified” refers to a cannabinoid (a “purified cannabinoid”) that is separated from secondary compounds that were present in the plant matter from which it was derived. In one embodiment, the term “purified” refers to a cannabinoid (a “purified cannabinoid”) that is separated from all material that was present in the plant matter from which it was derived.

In one embodiment, the term “purified” refers to a terpene (a “purified terpene”) that is separated from other cannabinoids that were present in the plant matter from which it was derived. In one embodiment, the term “purified” refers to a terpene (a “purified terpene”) that is separated from terpenes that were present in the plant matter from which it was derived. In one embodiment, the term “purified” refers to a terpene (a “purified terpene”) that is separated from secondary compounds that were present in the plant matter from which it was derived. In one embodiment, the term “purified” refers to a terpene (a “purified terpene”) that is separated from all material that was present in the plant matter from which it was derived.

Within the context of this disclosure, purified compounds may be purposely formulated with other compounds at various levels of purity. For example, depending on the desired outcome, a particular cannabinoid or terpene may be formulated with other molecules when it is 60-65% pure, 65-70% pure, 70-75% pure, 75-80% pure, 80-85% pure, 85-90% pure, 90-95% pure, 95-99% pure, 99-99.9% pure, 99.9+%, or greater than 99% pure. Provided that the ingredients used for purposeful formulation are purified prior to the said purposeful formulation, the act of subsequently formulating them does render them not “purified” within the context of an ingredient list.

In one embodiment, purified means “substantially free” from other material, e.g., compounds, particles, vegetative material, plant derived substances, solvents, etc. In one example, the term “purified” refers to a compound purified from a crude extract, such as a biologically derived substance or BDS, thereby resulting in a significant difference between the purified compound and the extract. In one embodiment, substantially free means that the compound, or composition containing the compound, comprises no (or insignificant amounts) of other materials. In another embodiment, substantially free means that the compound, or composition containing the compound, comprises less than 1%, less than 0.9%, less than 0.8%, less than 0.7%, less than 0.6%, less than 0.5%, less than 0.4%, less than 0.3%, less than 0.25%, less than 0.2%, less than 0.15%, less than 0.1%, or less than 0.05% of other materials.

Within the context of this disclosure, the term “plant” refers to the whole plant and parts of the plant, e.g., leaves, roots, bark, flower, etc. The term plant also encompasses a dried plant and dried parts of the plant.

Within the context of this disclosure, where a compound comprises stereogenic centers, the term “purified” includes isolated stereoisomers and also mixtures of stereoisomers, provided that the compound having the stereoisomers is free from other compounds having different atomic connectivity.

As used within the context of this application, the term “manmade” means engineered or purposefully created by a human in contrast to naturally “in nature” without human influence. In one embodiment, manmade compositions are formulations made by combining naturally occurring molecules in a new unnatural way, for example by separating the individual chemical components of a plant and then recombining some of those components to achieve different concentrations and/or ratios of those components. In one embodiment, manmade compositions are formulations made with one or more processes in a chemical extraction and purification lab. The properties of compositions of this disclosure—made by purposefully combining purified compounds with concentrations, combinations and/or ratios—differ from previously existing technology, such as extracts or other processed materials, e.g., biological drug substance, medical grade plant extracts, etc.

In one embodiment, purification comprises using various solvents, e.g., ethanol, butane, methane, carbon dioxide, ice, water, steam. In one embodiment, purification comprises various techniques, e.g., chromatography, crystallization, filtration, centrifuge, etc. or various combinations of said techniques. In one embodiment, purification comprises isolating cannabinoids and other plant molecules from plants bred to express desired cannabinoid and/or terpene profiles for purity.

As used herein, the term “cellulose” means an organic compound with the formula (C₆H₁₀O₅)ₙ. In one embodiment, the phrase “substantially free from cellulose” is intended to distinguish purified components of naturally occurring plants (for example the cannabis plant) from the isolated chemical compounds that are free from plant material, such as cellulose.

As used herein, the term “naturally occurring” refers to materializing, arising, happening, or synthesizing in nature. In one embodiment, naturally occurring refers to a secondary compound synthesized within a plant. In one embodiment, naturally occurring refers to a collection of cannabinoids synthesized within a plant of genus cannabis. In one embodiment, naturally occurring refers to the concentrations of compounds within a plant. In one embodiment, naturally occurring refers to the concentration of cannabinoids within a plant of genus cannabis. In one embodiment, naturally occurring refers to the ratio of secondary compounds within a plant. In one embodiment, the naturally occurring refers to the ratio of a first cannabinoid to a second cannabinoid within a plant of genus cannabis.

As used herein, the term “naturally occurring ratio” refers to the proportion of one compound or compounds in relation to another compound or compounds within a plant of genus cannabis. In one embodiment, the naturally occurring ratio is the amount of a cannabinoid in relation to the amount of a cannabinoid within a plant of genus cannabis. In one embodiment, the naturally occurring ratio is the amount of a cannabinoid in relation to the amount of a terpene within a plant of genus cannabis. In one embodiment, the naturally occurring ratio is the amount of a cannabinoid in relation to the amount of a terpene within a flower of genus cannabis. In one embodiment, the naturally occurring ratio is the amount of a cannabinoid in relation to the amount of a terpene within a plant extract of genus. In one embodiment, the naturally occurring ratio is the amount of a cannabinoid in relation to the amount of a terpene within a formulation made from plant extract of genus cannabis.

In one embodiment, the naturally occurring ratio is expressed as a molar ratio. In one embodiment, the naturally occurring ratio is expressed as a mass. In one embodiment, the mass and/or molar ratio is measured by chromatography and/or spectroscopy.

As used herein, the term “unnaturally occurring ratio” refers to the proportion of one compound or compounds in relation to another compound or compounds in a composition created by a human. In one embodiment, the unnaturally occurring ratio is the amount of a cannabinoid in relation to a terpene and is not observed in a plant of genus cannabis. In one embodiment, the unnaturally occurring ratio is expressed as a molar ratio. In one embodiment, the unnaturally occurring ratio is expressed as a mass. In one embodiment, the mass and/or molar ratio is measured by chromatography and/or spectroscopy.

In one embodiment, the unnaturally occurring ratio is the amount of a cannabinoid in relation to the amount of a terpene within a composition formulated by a human not observed in a plant of genus cannabis. In one embodiment, the unnaturally occurring ratio is the amount of a cannabinoid in relation to the amount of a second cannabinoid within a composition formulated by a human not observed in a plant of genus cannabis.

As used herein, the term “naturally occurring concentration” refers to the amount of a compound or compounds in relation to an entire naturally occurring reference sample. In one embodiment, the naturally occurring concentration is the amount of a cannabinoid in a sample of a plant of genus cannabis. In one embodiment, the naturally occurring concentration is the amount of a cannabinoid within the dried, or cured, flower of a plant of genus cannabis. In one embodiment, the naturally occurring concentration is the amount of a cannabinoid within a crude extract of a plant of genus cannabis.

In one embodiment, the naturally occurring concentration is the amount of a terpene in a sample of a plant of genus cannabis. In one embodiment, the naturally occurring concentration is the amount of a terpene within the dried, or cured, flower of a plant of genus cannabis. In one embodiment, the naturally occurring concentration is the amount of a terpene within a crude extract of a plant of genus cannabis. In one embodiment, the unnaturally occurring concentration is measured by moles and expressed as Molarity. In one embodiment, the naturally occurring concentration is measured by percent mass.

As used herein, the term “unnaturally occurring concentration” refers to the amount of a compound or compounds in relation to an entire sample within a manmade composition. In one embodiment, the unnaturally occurring concentration is the amount of a cannabinoid in relation to the total composition. In one embodiment, the unnaturally occurring concentration is the amount of a terpene in relation to the total composition. In one embodiment, the unnaturally occurring concentration is measured by moles and expressed as Molarity. In one embodiment, the unnaturally occurring concentration is measured by percent mass.

As used herein, the term “total mass” refers to the entire amount of matter for a given reference sample. In one embodiment, the total mass is measured by molecular mass.

The term “pharmacological fingerprint” refers to a collection of data about the activity of a single cannabinoid or terpene, or any combination of cannabinoids and/or terpenes, at one or more targets in the human body, e.g., a cannabinoid receptor, serotonin receptor, μ-opioid receptor, dopamine receptor, or GABA receptor, e.g., CB1, CB2, GPR55, 5HT-1A, 5HT-2A, TRPV1, etc. The compositions used in the methods disclosed herein were created using cellular (e.g., pharmacological fingerprint) and/or clinical data to select and combine particular combinations of compounds having new properties.

As used herein, the term “potency” refers to the power, influence, activity, or effectiveness of the compositions disclosed herein at a particular cellular receptor. In one embodiment, potency is measured by the response of a subject. In one embodiment, potency is measured by the cellular reactivity of a cannabinoid at a receptor, e.g., CB1, CB2, GPR55, 5HT-1A, 5HT-2A, TRPV1, etc. In one embodiment, potency refers to the EC50. In one embodiment, potency refers to amplitude of response.

As used herein, the term “agonism” refers to the effect of a compound, agonist, activating a receptor and inducing a response. In one embodiment, the receptor is chosen from a cannabinoid receptor, serotonin receptor, or μ-opioid receptor. In one embodiment the receptor is chosen from CB1, CB2, GPR55, 5HT-1A, 5HT-2A, or TRPV1. In one embodiment, the agonist is a cannabinoid. In one embodiment, the agonist is a terpene.

As used herein, the term “antagonism” refers to two or more compounds in combination having an effect. In one embodiment, antagonism is a composition comprising a cannabinoid affecting another cannabinoid, i.e., a cannabinoid affecting the binding of another cannabinoid to a receptor. In one embodiment, antagonism is a composition comprising a terpene affecting another cannabinoid, i.e., a terpene affecting the binding of another cannabinoid to a receptor.

For example, formulations of this disclosure have been achieved by expressing the human components of the endocannabinoid system (or other known targets of cannabis pharmacology) in cultured cells and treating the cells with serial dilutions of individual compounds or combinations of compounds in order to determine the EC50s of each component at each target individually, and whether adding other components shifts either the amplitude of the response or the effective concentration (EC50) of the response.

As used herein, the term “EC50” means “half maximal effective concentration,” which refers to the concentration of a formulation (e.g., a cannabinoid, terpene, or combination thereof) inducing a response halfway between the baseline and maximum after a specified exposure time. In one embodiment, the EC50 value is used as a measure of a drug's potency.

The EC50 of a graded dose response curve represents the concentration of a compound where 50% of its maximal effect is observed. The EC50 of a quantal dose response curve represents the concentration of a compound where 50% of the population exhibit a response, after a specified exposure duration.

As used herein, the term “amplitude of response” refers to the magnitude of reactivity to the compositions disclosed herein. In one embodiment, the amplitude of response is the reaction of a receptor, e.g., CB1, CB2, GPR55, 5HT-1A, 5HT-2A, TRPV1, serotonin receptor, or μ-opioid receptor, to a sample administered thereto.

For example, in one system, D9-THC activity at cannabinoid receptor 1 (CB1) has a consistent EC50 of around 1 micromolar, and a 40-50% amplitude of response compared to a known, synthetic full agonist (CP-55,940). The lower response of CB1 to THC relative to a full agonist is why THC is referred to as a “partial agonist” of the CB1 receptor.

In some embodiments, the purposefully engineered formulations disclosed herein were developed by establishing the EC50 of THC at CB1 numerous times and determining EC50s of THC in a background of constant concentrations of other cannabinoids and terpenes. This is one example of a method for determining how particular cannabis components may either synergize, add to, or compete with the THC response itself.

Using these purposefully engineered combinations (rather than naturally occurring combinations) provide a user with quantifiable control over the underlying pharmacology and biochemistry responsible for the observed physical and psychological effects. Accordingly, the compositions used in the methods disclosed herein provide unprecedented control over the effects of cannabinoids and/or terpenes on the user.

In one embodiment, the composition provides a decrease in the EC50 for either or both of a first purified cannabinoid and/or a second purified cannabinoid at a particular receptor.

In one embodiment, the composition provides an increase in the EC50 for either or both of the purified cannabinoid and/or a second purified cannabinoid at a particular receptor, for example the CB1, CB2 GPR55, 5HT-1A, 5HT-2A, TRPV1, and/or μ-opioid receptor.

In one embodiment, the purified terpene used in the methods disclosed herein is chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

Disclosed herein are methods of increasing the potency at a receptor, e.g. CB1, CB2 GPR55, 5HT-1A, 5HT-2A, TRPV1, serotonin receptor, μ-opioid receptor, etc., of a first purified cannabinoid in concert with a first purified terpene. In one embodiment, increasing the potency at a receptor comprises a second purified cannabinoid. In one embodiment, increasing the potency at a receptor comprises a second purified terpene.

In one embodiment, a purified cannabinoid, such as but not limited to cannabichromene (CBC), in combination with a purified terpene, such as but not limited to caryophyllene or alpha-terpinene, may elicit greater than 2-fold shifts in EC50 response at a CB2 receptor. In one embodiment, a purified cannabinoid and a purified terpene at unnaturally occurring ratios and/or unnaturally occurring concentrations provided substantially greater than 2-fold shifts in EC50 response at a CB2 receptor. In one embodiment, a purified cannabinoid in combination with a purified terpene may elicit a synergistic effect at a CB2 receptor.

In one embodiment, a purified cannabinoid, such as but not limited to cannabichromene (CBC), in combination with a combination of purified terpenes, such as caryophyllene and a second purified terpene may elicit greater than 2-fold shifts in EC50 response above a control featuring the cannabinoid and a single terpene such as caryophyllene. In one embodiment, a purified cannabinoid and the combination of purified terpenes at unnaturally occurring ratios and/or unnaturally occurring concentrations provided substantially greater than 2-fold shifts in EC50 response above a control featuring the cannabinoid and a single terpene such as caryophyllene.

In one embodiment, the compositions disclosed herein provide methods of increasing the response of THC at the CB1 receptor (relative to THC alone) comprising administering THC in concert with a terpene. In one example the method of increasing THC potency comprises administering THC in concert with alpha-humulene. In one example the method of increasing THC potency comprises administering THC in concert with linalool. In one example the method of increasing THC potency comprises administering THC in concert with nerolidol. In one example the method of increasing THC potency comprises administering THC in concert with limonene. In one example the method of increasing THC potency comprises administering THC in concert with alpha-terpineol. In one example the method of increasing THC potency comprises administering THC in concert with beta-pinene. In one example the method of increasing THC potency comprises administering THC in concert with p-cymene. In one example the method of increasing THC potency comprises administering THC in concert with eucalyptol.

In one embodiment, the compositions disclosed herein provide methods of increasing the response of THC at the CB1 receptor (relative to THC alone) comprising administering THC in concert with a second cannabinoid. In one example the method of increasing THC potency comprises administering THC in concert with CBC. In one example the method of increasing THC potency comprises administering THC in concert with CBG. In one example the method of increasing THC potency comprises administering THC in concert with THCV. In one example the method of increasing THC potency comprises administering THC in concert with CBD.

In one example, the disclosed compositions compete with THC at the CB1 receptor.

In one example, the EC50 of THC at CB1 is decreased (relative to THC alone) by formulating a composition comprising purified THC and a purified terpene chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

In one example, the EC50 of THC at CB1 is increased (relative to THC alone) by formulating a composition comprising purified THC and beta-caryophyllene.

In one embodiment of the compositions disclosed herein, the phrase “comprising a purified THC and a purified terpene” means that the composition includes between about 0.5% to about 10% combined terpene content.

An exemplary non-limiting terpene is linalool. One embodiment of this disclosure comprises 0.5-1% purified linalool. One embodiment of this disclosure comprises 1-2% purified linalool. One embodiment of this disclosure comprises 2-5% purified linalool. One embodiment of this disclosure comprises 5-10% purified linalool.

In one embodiment a purified composition comprising CBC is administered to a subject in need thereof for treatment of pain. Said CBC acts in accordance with the results disclosed herein as an agonist of the CB2 receptor, with potencies at least 4 times greater than previously seen with other phytocannaboid compounds such as CBD acting at the CB2 receptor.

In one embodiment a purified composition comprising CBC is administered to a subject in need thereof for reducing inflammation and/or treatment of inflammation and/or inflammatory disease. Said CBC acts in accordance with the results disclosed herein as an agonist of the CB2 receptor, with potencies at least 4 times greater than previously seen with other phytocannaboid compounds such as CBD acting at the CB2 receptor.

As used herein, the term “cannabinoid” refers to a compound belonging to a class of secondary compounds commonly found in plants of genus cannabis. In one embodiment, the cannabinoid is found in a plant, e.g., a plant of genus cannabis, and is sometimes referred to as a phytocannabinoid. In one embodiment, the cannabinoid is found in a mammal, sometimes called an endocannabinoid. In one embodiment, the cannabinoid is made in a laboratory setting, sometimes called a synthetic cannabinoid. In one embodiment, the cannabinoid acts upon a cellular receptor, such as a G-coupled protein receptor (e.g., a serotonin receptor, a cannabinoid receptor, TRPV1, an opioid receptor, etc.) thereby causing a response on the brain or body. In one embodiment, the cannabinoid affects the activity of other compounds at one or more receptors by acting as an agonist, partial agonist, inverse agonist, antagonist, etc.

In many cases, a cannabinoid can be identified because its chemical name will include the text string “*cannabi*” in the name.

Within the context of this application, where reference is made to a particular cannabinoid, each of the acid and/or decarboxylated forms are contemplated as both single molecules and mixtures.

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C₄ (CBD-C₄), Cannabidivarinic Acid (CBDVA), Cannabidivarin (CBDV), Cannabidiorcol (CBD-C₁), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C₄ (THCA-C₄), Tetrahydrocannbinol C₄ (THC-C₄), Tetrahydrocannabivarinic acid (THCVA), Tetrahydrocannabivarin (THCV), Tetrahydrocannabiorcolic acid (THCA-C₁), Tetrahydrocannabiorcol (THC-C₁), Δ⁷-cis-iso-tetrahydrocannabivarin, Δ⁸-tetrahydrocannabinolic acid (Δ⁸-THCA), Cannabivarinodiolic (CBNDVA), Cannabivarinodiol (CBNDV), Δ⁸-tetrahydrocannabinol (Δ⁸-THC), Δ⁹-tetrahydrocannabinol (Δ⁹-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA), Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C₄ (CBN-C₄), Cannabivarin (CBV), Cannabino-C₂ (CBN-C₂), Cannabiorcol (CBN-C₁), Cannabinodiol (CBND), Cannabinodiolic Acid (CBNDA), Cannabinodivarin (CBDV), Cannabitriol (CBT), 10-Ethoxy-9-hydroxy-Δ^{6a}-tetrahydrocannabinol, 8,9-Dihydroxy-Δ^{6a(10a)}-tetrahydrocannabinol (8,9-Di-OH-CBT-C₅), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ^{6a(10a)}-tetrahydrocannabinol (OTHC), Δ⁹-cis-tetrahydrocannabinoi (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, and Dodeca-2E,4E-dienoic acid isobutylamide.

In one embodiment, the purified cannabinoid is chosen from THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

In one embodiment, the purified cannabinoid that is used within the methods disclosed herein is CBC. Accordingly, disclosed herein are methods comprising administering purified CBC to a subject in need of CBC.

As used herein, the term “CBC” refers to cannabichromene and has the following structural formula:

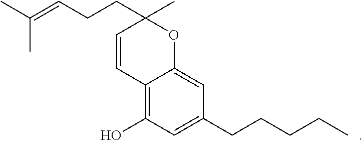 [SMILES: CCCCCc1cc(O)c2c(c1)OC(C)(CCC=C(C)C)C=C2]

In one embodiment of this disclosure, compositions comprising CBC are formulated with other compounds, thereby providing previously unavailable potency, control, consistency, purity, etc., for use in the methods disclosed herein. In one embodiment, the compositions disclosed herein comprise CBC as the only active ingredient. Accordingly, disclosed herein are formulating comprising purified CBC and methods of administering formulations comprising purified CBC.

As used herein, the term “CBCA” refers to cannabichromenic acid and has the following structural formula:

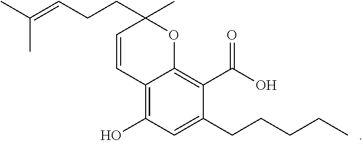 [SMILES: CCCCCc1cc(O)c2c(c1C(=O)O)OC(C)(CCC=C(C)C)C=C2]

Decarboxylating CBCA with heat, light, etc., forms CBC and other possible cannabinoid derivatives. In some embodiments of this disclosure, compositions comprising CBCA are formulated with other compounds, thereby providing previously unavailable potency, control, consistency, purity, etc. for use in the methods disclosed herein. In one embodiment of this disclosure, CBCA is used as a means of providing CBC, e.g., by decarboxylating CBCA.

As used herein, the term “CBCV” refers to cannabichromevarin and has the following structural formula:

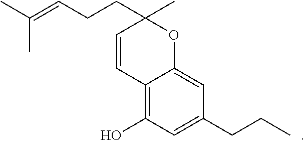 [SMILES: CCCc1cc(O)c2c(c1)OC(C)(CCC=C(C)C)C=C2]

Within the context of this disclosure, compositions comprising CBCV are formulated with other compounds, thereby providing previously unavailable potency, control, consistency, purity, etc.

As used herein, the term “CBCVA” refers to cannabichromevarinic acid and has the following structural formula:

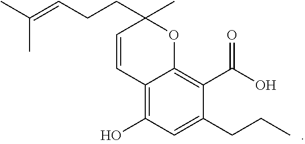 [SMILES: CCCc1cc(O)c2c(c1C(=O)O)OC(C)(CCC=C(C)C)C=C2]

Decarboxylating CBCVA with heat, light, etc., forms CBCV and other possible cannabinoid derivatives. Within the context of this disclosure, compositions comprising CBCVA are formulated with other compounds, thereby providing previously unavailable potency, control, consistency, purity, etc.

As used herein, the term “CBG” refers to cannabigerol and has the following structural formula:

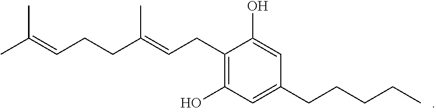 [SMILES: CCCCCc1cc(O)c(C/C=C(\C)CCC=C(C)C)c(O)c1]

Within the context of this disclosure, compositions comprising CBG are formulated with other compounds, thereby providing previously unavailable potency, control, consistency, purity, etc.

As used herein, the term “CBGA” refers to cannabigerolic acid and has the following structural formula:

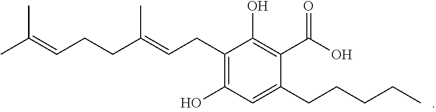 [SMILES: CCCCCc1cc(O)c(C/C=C(\C)CCC=C(C)C)c(O)c1C(=O)O]

Decarboxylating CBGA with heat, light, etc., forms CBG and other possible cannabinoid derivatives. Within the context of this disclosure, compositions comprising CBGA are formulated with other compounds, thereby providing previously unavailable potency, control, consistency, purity, etc.

As used herein, the term “CBGV” refers to cannabigerovarin and has the following structural formula:

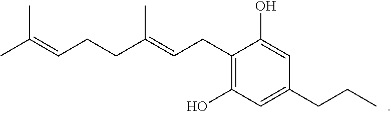 [SMILES: CCCc1cc(O)c(C/C=C(\C)CCC=C(C)C)c(O)c1]

Within the context of this disclosure, compositions comprising CBGV are formulated with other compounds, thereby providing previously unavailable potency, control, consistency, purity, etc.

As used herein, the term “CBGVA” refers to cannabigerovarinic acid and has the following structural formula:

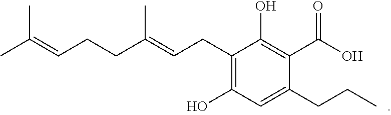 [SMILES: CCCc1cc(O)c(C/C=C(\C)CCC=C(C)C)c(O)c1C(=O)O]

Decarboxylating CBGVA with heat, light, etc., forms CBGV and other possible cannabinoid derivatives. Within the context of this disclosure, compositions comprising CBGVA are formulated with other compounds, thereby providing previously unavailable potency, control, consistency, purity, etc.

As used herein, the term “CBN” refers to cannabinol and has the following structural formula:

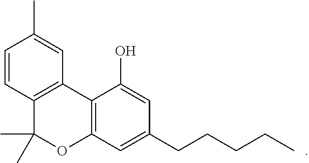 [SMILES: CCCCCc1cc(O)c2c(c1)OC(C)(C)c1ccc(C)cc1-2]

Within the context of this disclosure, compositions comprising CBN are formulated with other compounds, thereby providing previously unavailable potency, control, consistency, purity, etc.

As used herein, the term “CBNA” refers to cannabinolic acid and has the following structural formula:

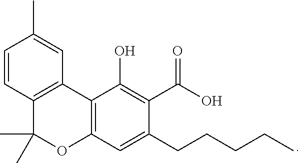 [SMILES: CCCCCc1cc2c(c(O)c1C(=O)O)-c1cc(C)ccc1C(C)(C)O2]

Decarboxylating CBNA with heat, light, etc., forms CBN and other possible cannabinoid derivatives. Within the context of this disclosure, compositions comprising CBNA are formulated with other compounds, thereby providing previously unavailable potency, control, consistency, purity, etc.

As used herein, the term “CBNV” or “CBV” refers to cannabivarin and has the following structural formula:

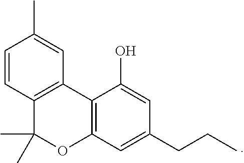 [SMILES: CCCc1cc(O)c2c(c1)OC(C)(C)c1ccc(C)cc1-2]

Within the context of this disclosure, compositions comprising CBNV are formulated with other compounds, thereby providing previously unavailable potency, control, consistency, purity, etc.

As used herein, the term “CBNVA” refers to cannabivarinic acid and has the following structural formula:

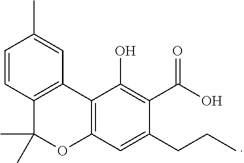 [SMILES: CCCc1cc2c(c(O)c1C(=O)O)-c1cc(C)ccc1C(C)(C)O2]

Decarboxylating CBNVA with heat, light, etc., forms CBNV and other possible cannabinoid derivatives. Within the context of this disclosure, compositions comprising CBNVA are formulated with other compounds, thereby providing previously unavailable potency, control, consistency, purity, etc.

As used herein, the term “CBND” refers to cannabinodiol and has the following structural formula:

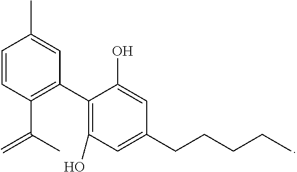 [SMILES: C=C(C)c1ccc(C)cc1-c1c(O)cc(CCCCC)cc1O]

Within the context of this disclosure, compositions comprising CBND are formulated with other compounds, thereby providing previously unavailable potency, control, consistency, purity, etc.

As used herein, the term “CBNDA” refers to cannabinodiolic acid and has the following structural formula:

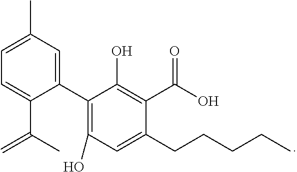 [SMILES: C=C(C)c1ccc(C)cc1-c1c(O)cc(CCCCC)c(C(=O)O)c1O]

Decarboxylating CBNDA with heat, light, etc., forms CBND and other possible cannabinoid derivatives. Within the context of this disclosure, compositions comprising CBNDA are formulated with other compounds, thereby providing previously unavailable potency, control, consistency, purity, etc.

As used herein, the term “CBNDV” refers to cannabivarinodiol and has the following structural formula:

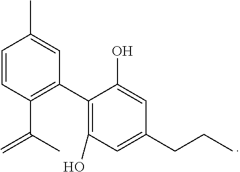 [SMILES: C=C(C)c1ccc(C)cc1-c1c(O)cc(CCC)cc1O]

Within the context of this disclosure, compositions comprising CBNDV are formulated with other compounds, thereby providing previously unavailable potency, control, consistency, purity, etc.

As used herein, the term “CBNDVA” refers to cannabivarinodiolic acid and has the following structural formula:

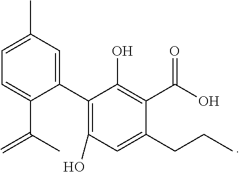 [SMILES: C=C(C)c1ccc(C)cc1-c1c(O)cc(CCC)c(C(=O)O)c1O]

Decarboxylating CBNDVA with heat, light, etc., forms CBNDV and other possible cannabinoid derivatives. Within the context of this disclosure, compositions comprising CBNDVA are formulated with other compounds, thereby providing previously unavailable potency, control, consistency, purity, etc.

As used herein, the term “CBL” refers to cannabicyclol and has the following structural formula:

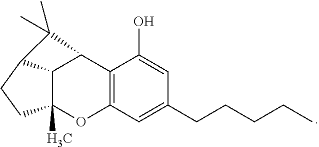

Within the context of this disclosure, compositions comprising CBL are formulated with other compounds, thereby providing previously unavailable potency, control, consistency, purity, etc.

As used herein, the term “CBLA” refers to cannabicyclolic acid and has the following structural formula:

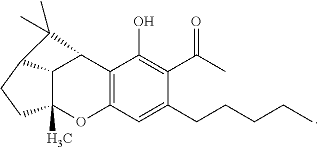

Decarboxylating CBLA with heat, light, etc., forms CBL and other possible cannabinoid derivatives. Within the context of this disclosure, compositions comprising CBLA are formulated with other compounds, thereby providing previously unavailable potency, control, consistency, purity, etc.

As used herein, the term “CBLV” refers to cannabicyclovarin and has the following structural formula:

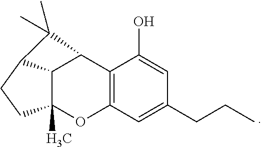

Within the context of this disclosure, compositions comprising CBLV are formulated with other compounds, thereby providing previously unavailable potency, control, consistency, purity, etc.

As used herein, the term “CBLVA” refers to cannabielvarinsoinic acid and has the following structural formula:

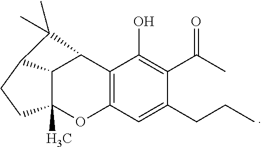

Decarboxylating CBLVA with heat, light, etc., forms CBLV and other possible cannabinoid derivatives. Within the context of this disclosure, compositions comprising CBLVA are formulated with other compounds, thereby providing previously unavailable potency, control, consistency, purity, etc.

As used herein, the term “CBE” refers to cannabielsoin and has the following structural formula:

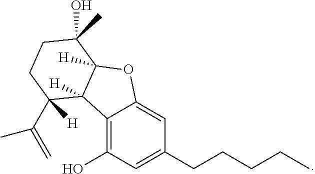

Within the context of this disclosure, compositions comprising CBE are formulated with other compounds, thereby providing previously unavailable potency, control, consistency, purity, etc.

As used herein, the term “CBEA” refers to cannabielsoic acid and has the following structural formula:

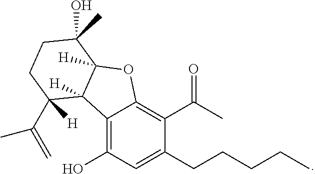

Decarboxylating CBEA with heat, light, etc., forms CBE and other possible cannabinoid derivatives. Within the context of this disclosure, compositions comprising CBEA are formulated with other compounds, thereby providing previously unavailable potency, control, consistency, purity, etc.

As used herein, the term “CBEV” refers to cannabivarinselsoin and has the following structural formula:

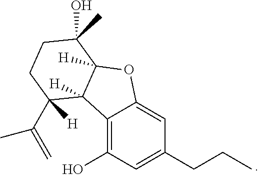

Within the context of this disclosure, compositions comprising CBEV are formulated with other compounds, thereby providing previously unavailable potency, control, consistency, purity, etc.

As used herein, the term “CBEVA” refers to cannabivarinselsoinic acid and has the following structural formula:

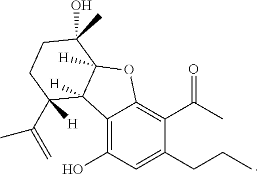

Decarboxylating CBEVA with heat, light, etc., forms CBEV and other possible cannabinoid derivatives. Within the context of this disclosure, compositions comprising CBEVA are formulated with other compounds, thereby providing previously unavailable potency, control, consistency, purity, etc.

As used herein, the term “terpene” refers to a compound built on an isoprenoid structure or produced by combining isoprene units, 5 carbon structures. Terpenes are also associated with producing smell in plants where terpenes are part of a class of secondary compounds. In one embodiment, the terpene is a hydrocarbon.

Within the context of this disclosure, the term “terpene” does not necessarily require 5 carbons or multiples of 5 carbons. It is understood that a reaction with isoprene units does not always result in a terpene comprising all the carbon atoms.

Within the context of this disclosure, the term “terpene” includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. As well as isomeric, enantiomeric, or optically active derivatives.

Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids.

Within the context of this disclosure, the term terpene includes the α- (alpha), β- (beta), γ- (gamma), oxo-, isomers, or any combinations thereof.

Examples of terpenes within the context of this disclosure include: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

In one embodiment, the disclosed compositions and methods comprising CBC additionally comprise a second purified cannabinoid chosen from THC, D9-THC, D8-THC, THCA, THCV, D8-THCV, D9-THCV, THCVA, CBD, CBDA, CBDV, CBDVA, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

Accordingly, disclosed herein are new compositions comprising unique combinations of CBC, a second purified cannabinoid, and a first purified terpene. In one embodiment, the compositions disclosed herein comprise CBC, a second purified cannabinoid, and a first purified terpene present in non-naturally occurring concentrations. In one embodiment, the compositions disclosed herein comprise CBC, a second purified cannabinoid, and a first purified terpene present in non-naturally occurring ratios. In one embodiment, the compositions disclosed herein provide particular benefits previously unavailable with naturally occurring cannabinoid profiles, such as those found in plants, harvested flowers, extracts, or conventional products derived from the same.

In one embodiment of the disclosed composition, the ratio of CBC to the second purified cannabinoid to the first purified terpene is greater than 100:1:1.

In one embodiment of the compositions disclosed herein, the ratio of CBC to the second purified cannabinoid to the first purified terpene is between 100:1:1 to 10:1:1.

In one embodiment of the compositions disclosed herein, the ratio of CBC to the second purified cannabinoid to the first purified terpene is between 20:1:1 to 10:1:1.

In one embodiment of the compositions disclosed herein, the ratio of CBC to the second purified cannabinoid to the first purified terpene is between 10:1:1 to 1:1:1.

In one embodiment of the compositions disclosed herein, the ratio of CBC to the second purified cannabinoid to the first purified terpene is between 5:1:1 to 1:1:1.

In one embodiment of the compositions disclosed herein, the ratio of CBC to the second purified cannabinoid to the first purified terpene is between 3:1:1 to 1:1:1.

In one embodiment of the compositions disclosed herein, the ratio of CBC to the second purified cannabinoid to the first purified terpene is between 1:1:1 to 1:1:10.

In one embodiment of the compositions disclosed herein, the ratio of CBC to the second purified cannabinoid to the first purified terpene is between 1:1:10 to 1:1:100.

In one embodiment of the compositions disclosed herein, the ratio of CBC to the second purified cannabinoid to the first purified terpene is between 100:50:1 to 10:5:1.

In one embodiment of the compositions disclosed herein, the ratio of CBC to the second purified cannabinoid to the first purified terpene is between 50:10:1 to 5:2:1.

In one embodiment of the compositions disclosed herein, the ratio of CBC to the second purified cannabinoid to the first purified terpene is between 25:15:1 to 1:1:1.

In one embodiment of the compositions disclosed herein, the ratio of CBC to the second purified cannabinoid to the first purified terpene is between 1:1:1 to 1:5:5.

In one embodiment of the compositions disclosed herein, the ratio of CBC to the second purified cannabinoid to the first purified terpene is between 1:5:5 to 1:25:100.

In one embodiment of the compositions disclosed herein, the ratio of CBC to the second purified cannabinoid to the first purified terpene is between 100:100:1 to 10:10:1.

In one embodiment of the compositions disclosed herein, the ratio of CBC to the second purified cannabinoid to the first purified terpene is between 20:20:1 to 10:10:1.

In one embodiment of the compositions disclosed herein, the ratio of CBC to the second purified cannabinoid to the first purified terpene is between 10:10:1 to 1:1:1.

In one embodiment of the compositions disclosed herein, the ratio of CBC to the second purified cannabinoid to the first purified terpene is between 5:5:1 to 1:1:1.

In one embodiment of the compositions disclosed herein, the ratio of CBC to the second purified cannabinoid to the first purified terpene is between 3:3:1 to 1:1:1.

In one embodiment of the compositions disclosed herein, the ratio of CBC to the second purified cannabinoid to the first purified terpene is between 1:1:1 to 1:10:10.

In one embodiment of the compositions disclosed herein, the ratio of CBC to the second purified cannabinoid to the first purified terpene is between 1:10:10 to 1:100:100.

In one embodiment of the disclosed composition, CBC, the second purified cannabinoid, and the first purified terpene, together, comprise between 80-100% of the total mass of the composition.

In one embodiment of the disclosed composition, CBC, the second purified cannabinoid, and the first purified terpene, together, comprise between 85-100% of the total mass of the composition.

In one embodiment of the disclosed composition, CBC, the second purified cannabinoid, and the first purified terpene, together, comprise between 90-100% of the total mass of the composition.

In one embodiment of the disclosed composition, CBC, the second purified cannabinoid, and the first purified terpene, together, comprise between 95-100% of the total mass of the composition.

In one embodiment of the disclosed composition, CBC, the second purified cannabinoid, and the first purified terpene, together, comprise between 99-100% of the total mass of the composition.

In one embodiment, the above described combinations of a first purified cannabinoid and a first purified terpene include a second purified terpene chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

Accordingly, disclosed herein are new compositions comprising unique combinations of a first purified cannabinoid, a first purified terpene, and a second purified terpene are present in non-naturally occurring concentrations. In one embodiment, CBC, the first purified terpene, and second purified terpene are present in non-naturally occurring ratios. In one embodiment, the compositions disclosed herein provide particular benefits previously unavailable with naturally occurring cannabinoid profiles, such as those found in plants, harvested flowers, extracts, or conventional products derived from the same.

In one embodiment of the disclosed composition, the ratio of CBC to the first purified terpene to the second purified terpene is greater than 100:1:1.

In one embodiment of the compositions disclosed herein, the ratio of CBC to the first purified terpene to the second purified terpene is between 100:1:1 to 10:1:1.

In one embodiment of the compositions disclosed herein, the ratio of CBC to the first purified terpene to the second purified terpene is between 20:1:1 to 10:1:1.

In one embodiment of the compositions disclosed herein, the ratio of CBC to the first purified terpene to the second purified terpene is between 10:1:1 to 1:1:1.

In one embodiment of the compositions disclosed herein, the ratio of CBC to the first purified terpene to the second purified terpene is between 5:1:1 to 1:1:1.

In one embodiment of the compositions disclosed herein, the ratio of CBC to the first purified terpene to the second purified terpene is between 3:1:1 to 1:1:1.

In one embodiment of the compositions disclosed herein, the ratio of CBC to the first purified terpene to the second purified terpene is between 1:1:1 to 1:1:10.

In one embodiment of the compositions disclosed herein, the ratio of CBC to the first purified terpene to the second purified terpene is between 1:1:10 to 1:1:100.

In one embodiment of the compositions disclosed herein, the ratio of CBC to the first purified terpene to the second purified terpene is between 100:50:1 to 10:5:1.

In one embodiment of the compositions disclosed herein, the ratio of CBC to the first purified terpene to the second purified terpene is between 50:10:1 to 5:2:1.

In one embodiment of the compositions disclosed herein, the ratio of CBC to the first purified terpene to the second purified terpene is between 25:15:1 to 1:1:1.

In one embodiment of the compositions disclosed herein, the ratio of CBC to the first purified terpene to the second purified terpene is between 1:1:1 to 1:5:5.

In one embodiment of the compositions disclosed herein, the ratio of CBC to the first purified terpene to the second purified terpene is between 1:5:5 to 1:25:100.

In one embodiment of the compositions disclosed herein, the ratio of CBC to the first purified terpene to the second purified terpene is between 100:100:1 to 10:10:1.

In one embodiment of the compositions disclosed herein, the ratio of CBC to the first purified terpene to the second purified terpene is between 20:20:1 to 10:10:1.

In one embodiment of the compositions disclosed herein, the ratio of CBC to the first purified terpene to the second purified terpene is between 10:10:1 to 1:1:1.

In one embodiment of the compositions disclosed herein, the ratio of CBC to the first purified terpene to the second purified terpene is between 5:5:1 to 1:1:1.

In one embodiment of the compositions disclosed herein, the ratio of CBC to the first purified terpene to the second purified terpene is between 3:3:1 to 1:1:1.

In one embodiment of the compositions disclosed herein, the ratio of CBC to the first purified terpene to the second purified terpene is between 1:1:1 to 1:10:10.

In one embodiment of the compositions disclosed herein, the ratio of CBC to the first purified terpene to the second purified terpene is between 1:10:10 to 1:100:100.

In one embodiment of the disclosed composition, the ratio of CBC to the first purified terpene to the second purified terpene, together, comprise between 80-100% of the total mass of the composition.

In one embodiment of the disclosed composition, the ratio of CBC to the first purified terpene to the second purified terpene, together, comprise between 85-100% of the total mass of the composition.

In one embodiment of the disclosed composition, the ratio of CBC to the first purified terpene to the second purified terpene, together, comprise between 90-100% of the total mass of the composition.

In one embodiment of the disclosed composition, the ratio of CBC to the first purified terpene to the second purified terpene, together, comprise between 95-100% of the total mass of the composition.

In one embodiment of the disclosed composition, the ratio of CBC to the first purified terpene to the second purified terpene, together, comprise between 99-100% of the total mass of the composition.

Disclosed herein are new compositions comprising combinations of a first purified cannabinoid and a second purified cannabinoid. In one embodiment, compositions disclosed herein are manmade. In one embodiment, CBC and the second purified cannabinoid are present in non-naturally occurring concentrations. In one embodiment, CBC and the second purified cannabinoid are present in non-naturally occurring ratios. In one embodiment, the compositions disclosed herein provide particular benefits previously unavailable with naturally occurring cannabinoid profiles, such as those found in plants, harvested flowers, extracts, or conventional products derived from the same.

In one embodiment, the said combination of a first purified cannabinoid and a second purified cannabinoid provide a synergistic effect at a mammalian cellular receptor, such as a G protein coupled receptor. In one embodiment, the receptor is CB1. In one embodiment, the receptor is CB2. In one embodiment, the receptor is GPR55. In one embodiment, the receptor is 5HT-1A.

In one embodiment, the receptor is 5HT-2A.

In one embodiment of the compositions disclosed herein, the ratio of CBC to the second purified cannabinoid is greater than 100:1.

In one embodiment of the compositions disclosed herein, the ratio of CBC to the second purified cannabinoid is between 100:1 to 10:1.

In one embodiment of the compositions disclosed herein, the ratio of CBC to the second purified cannabinoid is between 20:1 to 10:1.

In one embodiment of the compositions disclosed herein, the ratio of CBC to the second purified cannabinoid is between 10:1 to 1:1.

In one embodiment of the compositions disclosed herein, the ratio of CBC to the second purified cannabinoid is between 5:1 to 1:1.

In one embodiment of the compositions disclosed herein, the ratio of CBC to the second purified cannabinoid is between 3:1 to 1:1.

In one embodiment of the compositions disclosed herein, the ratio of CBC to the second purified cannabinoid is between 1:1 to 1:10.

In one embodiment of the compositions disclosed herein, CBC and the second purified cannabinoid comprise between 80-100% of the total mass of the composition.

In one embodiment of the compositions disclosed herein, CBC and the second purified cannabinoid comprise between 85-100% of the total mass of the composition.

In one embodiment of the compositions disclosed herein, CBC and the second purified cannabinoid comprise between 90-100% of the total mass of the composition.

In one embodiment of the compositions disclosed herein, CBC and the second purified cannabinoid comprise between 95-100% of the total mass of the composition.

In one embodiment of the compositions disclosed herein, CBC and the second purified cannabinoid comprise between 99-100% of the total mass of the composition.

In one embodiment of the compositions disclosed herein, the first purified cannabinoid is CBC and the second purified cannabinoid is CBCA.

In one embodiment of the compositions disclosed herein, the first purified cannabinoid is CBC and the second purified cannabinoid is CBCV.

In one embodiment of the compositions disclosed herein, the first purified cannabinoid is CBC and the second purified cannabinoid is CBCVA.

In one embodiment of the compositions disclosed herein, the first purified cannabinoid is CBC and the second purified cannabinoid is CBG.

In one embodiment of the compositions disclosed herein, the first purified cannabinoid is CBC and the second purified cannabinoid is CBGA.

In one embodiment of the compositions disclosed herein, the first purified cannabinoid is CBC and the second purified cannabinoid is CBGV.

In one embodiment of the compositions disclosed herein, the first purified cannabinoid is CBC and the second purified cannabinoid is CBGVA.

In one embodiment of the compositions disclosed herein, the first purified cannabinoid is CBC and the second purified cannabinoid is CBN.

In one embodiment of the compositions disclosed herein, the first purified cannabinoid is CBC and the second purified cannabinoid is CBNA.

In one embodiment of the compositions disclosed herein, the first purified cannabinoid is CBC and the second purified cannabinoid is CBNV.

In one embodiment of the compositions disclosed herein, the first purified cannabinoid is CBC and the second purified cannabinoid is CBNVA.

In one embodiment, the above described combinations of CBC and a second purified cannabinoid include a third purified cannabinoid chosen from THC, D9-THC, D8-THC, THCA, THCV, D8-THCV, D9-THCV, THCVA, CBD, CBDA, CBDV, CBDVA, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

Accordingly, disclosed herein are new compositions comprising unique combinations of CBC, a second purified cannabinoid, and a third purified cannabinoid. In one embodiment, the compositions disclosed herein comprise a first purified cannabinoid, a second purified cannabinoid, and a third purified cannabinoid present in non-naturally occurring concentrations. In one embodiment, the compositions disclosed herein comprise a first purified cannabinoid, a second purified cannabinoid, and a third purified cannabinoid present in non-naturally occurring ratios. In one embodiment, the compositions disclosed herein provide particular benefits previously unavailable with naturally occurring cannabinoid profiles, such as those found in plants, harvested flowers, extracts, or conventional products derived from the same.

In one embodiment of the compositions disclosed herein, the ratio of CBC to the second purified cannabinoid to the third purified cannabinoid is greater than 100:1:1.

In one embodiment of the compositions disclosed herein, the ratio of CBC to the second purified cannabinoid to the third purified cannabinoid is between 100:1:1 to 10:1:1.

In one embodiment of the compositions disclosed herein, the ratio of CBC to the second purified cannabinoid to the third purified cannabinoid is between 20:1:1 to 10:1:1.

In one embodiment of the compositions disclosed herein, the ratio of CBC to the second purified cannabinoid to the third purified cannabinoid is between 10:1:1 to 1:1:1.

In one embodiment of the compositions disclosed herein, the ratio of CBC to the second purified cannabinoid to the third purified cannabinoid is between 5:1:1 to 1:1:1.

In one embodiment of the compositions disclosed herein, the ratio of CBC to the second purified cannabinoid to the third purified cannabinoid is between 3:1:1 to 1:1:1.

In one embodiment of the compositions disclosed herein, the ratio of CBC to the second purified cannabinoid to the third purified cannabinoid is between 1:1:1 to 1:1:10.

In one embodiment of the compositions disclosed herein, the ratio of CBC to the second purified cannabinoid to the third purified cannabinoid is between 1:1:10 to 1:1:100.

In one embodiment of the compositions disclosed herein, the ratio of CBC to the second purified cannabinoid to the third purified cannabinoid is between 100:50:1 to 10:5:1.

In one embodiment of the compositions disclosed herein, the ratio of CBC to the second purified cannabinoid to the third purified cannabinoid is between 50:10:1 to 5:2:1.

In one embodiment of the compositions disclosed herein, the ratio of CBC to the second purified cannabinoid to the third purified cannabinoid is between 25:15:1 to 1:1:1.

In one embodiment of the compositions disclosed herein, the ratio of CBC to the second purified cannabinoid to the third purified cannabinoid is between 1:1:1 to 1:5:5.

In one embodiment of the compositions disclosed herein, the ratio of CBC to the second purified cannabinoid to the third purified cannabinoid is between 1:5:5 to 1:25:100.

In one embodiment of the compositions disclosed herein, the ratio of CBC to the second purified cannabinoid to the third purified cannabinoid is between 100:100:1 to 10:10:1.

In one embodiment of the compositions disclosed herein, the ratio of CBC to the second purified cannabinoid to the third purified cannabinoid is between 20:20:1 to 10:10:1.

In one embodiment of the compositions disclosed herein, the ratio of CBC to the second purified cannabinoid to the third purified cannabinoid is between 10:10:1 to 1:1:1.

In one embodiment of the compositions disclosed herein, the ratio of CBC to the second purified cannabinoid to the third purified cannabinoid is between 5:5:1 to 1:1:1.

In one embodiment of the compositions disclosed herein, the ratio of CBC to the second purified cannabinoid to the third purified cannabinoid is between 3:3:1 to 1:1:1.

In one embodiment of the compositions disclosed herein, the ratio of CBC to the second purified cannabinoid to the third purified cannabinoid is between 1:1:1 to 1:10:10.

In one embodiment of the compositions disclosed herein, the ratio of CBC to the second purified cannabinoid to the third purified cannabinoid is between 1:10:10 to 1:100:100.

### Examples

The following examples are provided as non-limiting illustrations of the broader disclosure.

### Materials and Methods: Assaying for Cannabinoid Receptor 2 (CB2) Activity

Modified versions of CHO-K1 cells were used, engineered to specifically express human CB2. Human CB2 was co-expressed in a stable cell line with the promiscuous G-protein G15, effectively coupling CB2 to the calcium pathway.

Dose plates were made in aqueous buffer using a Biomek liquid handling robot, at 5× the final assay concentration. For each day's batch of plates to be run, at least one control dose response is assayed, such as the synthetic cannabinoid CP55,940. Dose-responses of cannabichromene and other cannabinoids and terpenes are arrayed in triplicate on the dose plates, with 8 concentrations, in 3-fold serial dilutions.

Cells are grown in T75 or T150 flasks, and routinely harvested for propagation and for seeding assay plates. 96-well assay plates are seeded, typically, at 50,000 cells per well using a Multiflo dispenser (BioTek). Seeded assay plates were incubated at 37° C. overnight in a CO₂ incubator, at 5% CO₂, then incubated for 30 minutes with Fluo-4NW dye (Thermo-Fisher) according to the manufacturer's instructions. Twenty-five (25) microliters (1) of each well on the 5× dose plates was transferred robotically using the FLIPR (Molecular Devices) to assay plates containing 100 mL of media and dye. Responses were monitored on the FLIPR for several minutes at a rate of once per second. Each well generated a response, and the -RFU (change in relative fluorescence units) was extracted from each data trace. The -RFU values were plotted in a semi-log dose-response plot and fit to a four-parameter sigmoidal curve (GraphPad Prism) for determination of the EC50 for any individual compound or combination of compounds.

In the case of allosteric modulators, EC50s of the orthosteric ligand, such as cannabichromene, were measured in a background of saturating concentrations of the putative allosteric modulator(s) (typically 10 μM). Fold-shifts in the orthosteric ligand EC50 were calculated based on the control reaction from the same day's run.

Results indicate that a purified cannabinoid, such as but not limited to cannabichromene (CBC), in combination with a purified terpene, such as but not limited to caryophyllene or alpha-terpinene, can elicit greater than 2-fold shifts in EC50 response. Particular experiments that featured the purified cannabinoid and the purified terpene at unnaturally occurring ratios and/or unnaturally occurring concentrations provided substantially greater than 2-fold shifts in EC50 response. Results indicate synergistic effects with combinations.

Results indicate that a purified cannabinoid, such as but not limited to cannabichromene (CBC), in combination with a combination of purified terpenes, such as caryophyllene and a second purified terpene can elicit greater than 2-fold shifts in EC50 response above a control featuring the cannabinoid and a single terpene such as caryophyllene. Particular experiments that featured the purified cannabinoid and the combination of purified terpenes at unnaturally occurring ratios and/or unnaturally occurring concentrations provided substantially greater than 2-fold shifts in EC50 response above a control featuring the cannabinoid and a single terpene such as caryophyllene.

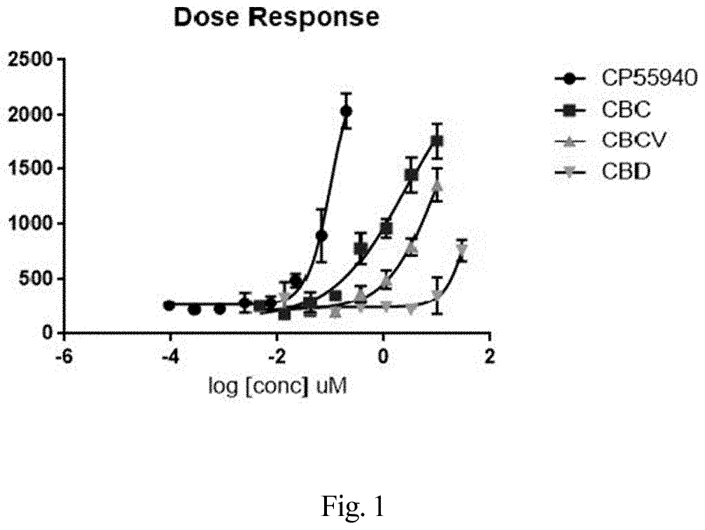

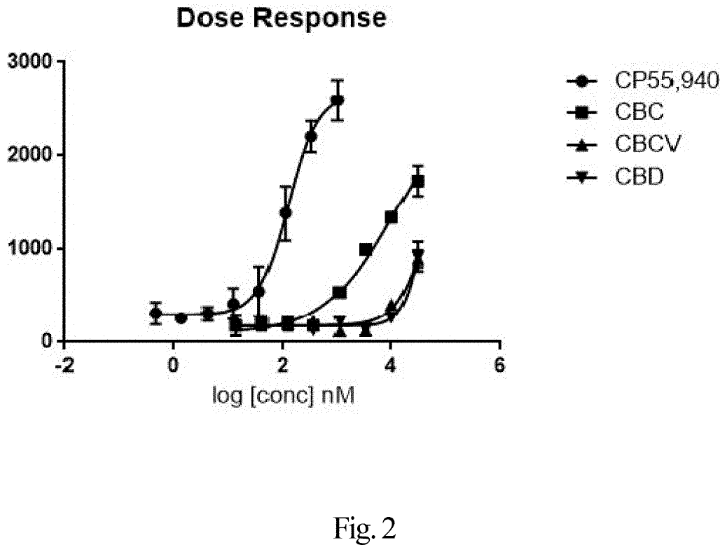

Data plots from two independent experiments (n=1 and n=2, respectively) are shown in FIG. 1 and FIG. 2, respectively.

CP55,940 was included as an internal control, as a known CB2 agonist that is 40-50× more potent than THC.

The plotted data clearly demonstrate that cannabichromene (CBC) is a more potent agonist at the human CB2 receptor than other phytocannabinoid agonists identified and studied previously, including cannabidiol (CBD), and cannabichromevarin (CBCV). In fact, CBC appears to exhibit at least 4-fold greater potency than CBD.

CBD is a known agonist at the CB2 receptor and is thought to exert many of its medical benefits through action at CB2. CB2 is a well-documented anti-inflammatory target. The results disclosed herein show that under the parameters assayed CBC is a significantly better anti-inflammatory compound than CBD. Accordingly, CBC may be used in treatments involving CB2 receptors that are free of THC, for example, in the treatment of inflammation and inflammatory diseases.

The CB2 receptor action of CBC also underscores the utility of CBC in the treatment of pain. Still further, CBC is also a known agonist at several transient receptor potential (TRP) channels, albeit somewhat weakly. Many TRP channels are nodal points of nociception, or the perception of pain. Thus, in accordance with the results disclosed herein, CBC is useful in the treatment of pain, and its actions may result via multiple mechanisms of action.

The examples used in this disclosure are meant to be non-limiting and serve only to illustrate possible embodiments of the formulations disclosed herein.

Although the present invention herein has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

What is claimed is:

1. A composition comprising:
purified CBC;
wherein the composition is substantially free from cellulose; and
wherein the purified CBC makes up greater than 50% of the total cannabinoids present in the composition.

2. The composition of claim 1, further comprising a compound chosen from a second purified cannabinoid, a purified terpene, and an analgesic.

3. The composition of claim 2, further comprising a purified terpene chosen from Linalool, Borneol, Elemene, Beta-Myrcene, Nerolidol, Phytol, Terpinolene, Bornyl Acetate, and Terpineol, alpha bisabolol.

4. The composition of claim 2, further comprising an analgesic.

5. The composition of claim 4, wherein the analgesic is chosen from Morphine, Thebaine, Buprenorphine, Fentanyl, Acetylfentanyl, Norfentanyl, 3-Methylfentanyl, Carfentanil, Alfentanil, Oxymorphone, Noroxymorphone, Hydromorphone, Tapentadol, Oxycodone, Hydrocodone, eroin, Diacetylmorphine, Codeine, Meperidine, Dextropropoxyphene, Papaverine, Levacetylmethado, Tramadol, Levorphanol, Methadone, Butorphanol, Pentazocine, and Nalbuphine.

6. A method of agonizing the CB2 receptor comprising administering a composition comprising purified CBC to a subject in need thereof.

7. A method of treating pain comprising administering a composition comprising purified CBC to a subject in need thereof.

8. The method of claim 7, further comprising administering an analgesic.

9. The method of claim 8, wherein the analgesic is chosen from chosen from Morphine, Thebaine, Buprenorphine, Fentanyl, Acetylfentanyl, Norfentanyl, 3-Methylfentanyl, Carfentanil, Alfentanil, Oxymorphone, Noroxymorphone, Hydromorphone, Tapentadol, Oxycodone, Hydrocodone, Heroin, Diacetylmorphine, Codeine, Meperidine, Dextropropoxyphene, Papaverine, Levacetylmethado, Tramadol, Levorphanol, Methadone, Butorphanol, Pentazocine, and Nalbuphine.

10. The method of claim 9, comprising reducing the amount of analgesic administered to the subject compared to the amount of analgesic required in the absence of CBC.

11. A method of reducing inflammation comprising administering a composition comprising purified CBC to a subject in need thereof.

12. A method of treating inflammatory disease comprising administering a composition comprising purified CBC to a subject in need thereof.
